(19) United States
(12) Patent Application Publication
(10) Pub. No.: US 2023/0114337 A1
(43) Pub. Date: Apr. 13, 2023

(54) ALTERNATIVE CANNULA CONFIGURATIONS TO CONTROL FLUID DISTRIBUTION IN TISSUE

(71) Applicant: Merck Sharp & Dohme LLC, Rahway, NJ (US)

(72) Inventors: Scott BROWN, Princeton, NJ (US); John B. CLINE, Monroe, NJ (US); Michael GALLUPPI, Bridgewater, NJ (US); Jeffrey C. GIVAND, Westfield, NJ (US); Guangli HU, Berkeley Heights, NJ (US); Steven Carl PERSAK, Morris Plains, NJ (US); Wail RASHEED, Union, NJ (US)

(73) Assignee: Merck Sharp & Dohme LLC, Rahway (US)

(21) Appl. No.: 17/798,754

(22) PCT Filed: Feb. 9, 2021
(86) PCT No.: PCT/US2021/017160

§ 371 Date: Aug. 10, 2022

Related U.S. Application Data
(60) Provisional application No. 62/972,869, filed on Feb. 11, 2020.

(51) Int. Cl. A61M 5/32; A61M 5/46

(52) U.S. Cl. CPC A61M 5/3291; A61M 5/46

(57) ABSTRACT

A cannula for subcutaneous dispensing of medicaments, wherein the cannula has a cannula wall, a distal end and a proximal end and wherein the cannula comprises a single orifice in the cannula wall, proximal to the distal end of the cannula or two or more orifices in the cannula wall, proximal to the distal end of the cannula.

### TECHNICAL FIELD

The present invention is directed to a cannula for subcutaneous injection of a medicament. Specifically, the cannula described herein is designed to subcutaneously inject a high dose of one or more medicaments into a patient through at least one orifice located on the cannula wall.

### BACKGROUND

The last decade has shown notable increases in the number of biological products (e.g. monoclonal antibodies), developed as parenteral formulations and advancing through the clinical development pipelines of most biopharmaceutical companies. These products span several therapeutic areas including auto immune, metabolic and oncology, resulting in a mixture of acute and chronic treatment regimens. Furthermore, while some products are best administered via intravenous (IV) infusion by a healthcare professional, others are being developed as formulations amenable to subcutaneous (SC) administration. The SC route of administration affords the benefits of a reduction in total treatment time for the patient, as well as the potential for patient self-administration in the home rather than a clinical setting.

Many of the monoclonal antibody formulations require moderate to high SC doses (100 mg-1000 mg) and have maximum allowable solution concentrations before physical stability limits are encountered. As a result, large injection volumes (greater than 2 ml) are being targeted for many of these formulations. Back pressure limitations can develop in the subcutaneous tissue for larger volumes being injected at typical rates causing either potential pain or leakage from the injection site. One option to manage these concerns would be to reduce the injection rate which can be difficult to manage using a standard syringe format. Sophisticated pumps (relative to a simple manual syringe) capable of slowing the injection rate to 1 mL/min or slower have been considered. More recently, chemical approaches have been developed whereby the biological product of interest is dosed with small quantities of recombinant human hyaluronidase PH20 enzyme which serves to locally degrade the hyaluronan increasing dispersion of the injectate and decreasing injection times.

Additionally, U.S. Pat. No. 8,333,734 discloses a method and apparatus for facilitating peripheral nerve block procedures comprising a needle with a plurality of fenestrations that enable local anesthetic to be administered simultaneously at several points surrounding an affected nerve. This approach provides a number of flow paths that are located around a single focal point. In contrast, the invention disclosed is designed to provide wide distribution of medicament along the length of the cannula.

The present invention proposes various designs to physically or mechanically disperse the injectable volume with the aim of minimizing local high subcutaneous pressure which would otherwise potentially produce pain or injection size leakage. The result of these approaches would permit delivery of larger volumes (>2 mL) in shorter injection times, with lower injection forces and utilizing simple and standard needle and syringe options. By properly sizing and spacing holes along the length of a standard subcutaneous needle, tuning the volumetric flow rate and fluid velocities from each orifice to more broadly distribute the injected volume relative to the traditional hypodermic needle with a single orifice is possible.

### SUMMARY

Described herein are cannulas for subcutaneous dispensing of medicaments, wherein the cannula has a cannula wall, a distal end and a proximal end and wherein the cannula comprises a single orifice in the cannula wall, proximal to the distal end of the cannula or two or more orifices in the cannula wall, proximal to the distal end of the cannula.

In certain embodiments, the cannula has a single orifice in the cannula wall, proximal to the distal end of the cannula.

In certain embodiments, the cannula comprises a single orifice in the cannula wall, proximal to the distal end of the cannula or two or more orifices in the cannula wall, proximal to the distal end of the cannula, wherein the orifice or two or more orifices are in the shape of a circle. In certain embodiments, the cannula has a single orifice in the cannula wall, proximal to the distal end of the cannula, wherein the orifice is in the shape of a circle. In certain embodiments, the cannula comprises two or more orifices in the cannula wall, proximal to the distal end of the cannula, wherein the two or more orifices are in the shape of a circle.

In certain embodiments, wherein the cannula has a single orifice in the cannula wall, proximal to the distal end of the cannula and wherein the orifice has a long axis, the long axis of the single orifice is not parallel to the axis of the cannula. In certain embodiments, the single orifice is oriented at an angle to the axis of the cannula between 5° and 90°.

In certain embodiments, wherein the cannula has two or more orifices in the cannula wall, proximal to the distal end of the cannula and wherein the two or more orifices have long axes, the long axes of each of the two or more orifices are not parallel to the axis of the cannula. In certain embodiments, the two or more orifices are oriented at an angle to the axis of the cannula between 5° and 90°.

In certain embodiments, the cannulas described herein is removably attached to a syringe. In certain embodiments, the removeable connection between the cannula and the syringe is a Luer connection. In other embodiments, the removeable connection between the cannula and the syringe is a threaded connection.

In certain embodiments, the cannula described herein is an integral part of the syringe.

In certain embodiments, the single orifice or at least one of the two or more is in the shape of an ellipse. In other embodiments, the single orifice or the or two or more orifices is in the shape of a polygon. In certain embodiments, the polygon is an elongated polygon. In certain embodiments, the two or more orifices comprise a combination of elliptical, and polygonal shapes. In certain embodiments, the length of the elongated polygon spans range between 1% and 80% of the length of the cannula. In certain embodiments, the long axis of the elongated polygon is oriented at an angle to the axis of the cannula between 0° and 45°.

In certain embodiments of the cannulas described herein, the single orifice or two or more orifices are not closed such that a portion of the perimeter of the opening of the orifice or two or more orifices intersects with the distal end of the cannula.

In certain embodiments of the cannulas described herein, the two or more orifices are oriented at a range of angles from one another, wherein the angles range from 0° to 180° when viewed in cross section along the axis of the cannula.

In certain embodiments of the cannulas described herein, the two or more orifices are spaced an unequal distance apart along the axis of the cannula. In certain embodiments, the two or more orifices are spaced at increasing distances apart in the distal direction along the axis of the cannula. In other embodiments, the two or more orifices are spaced at decreasing distances apart in the distal direction along the axis of the cannula.

In certain embodiments of the cannulas described herein, the single orifice or the two or more orifices each comprise an area between 0.000001 sq. in to 0.015 sq. in.

In certain embodiments, the cannula of the embodiments described herein, is tapered. In certain embodiments, the inner diameter of the cannula increases from the distal end of the cannula to the proximal end. In other embodiments, the inner diameter of the cannula increases from the distal end of the cannula to the proximal end, wherein the increase in inner diameter occurs along the entire length of the cannula. In still other embodiments, the inner diameter of the cannula increases from the distal end of the cannula to the proximal end, wherein the increase in inner diameter occurs along one or more discrete portions of the length of the cannula.

In certain embodiments of the cannulas described herein, the cannula further comprises a guide for controlling the angle and depth of insertion of the cannula.

In certain embodiments of the cannulas described herein, the guide limits the range of insertion depths of the distal end of the cannula between ¼ inches and 3 inches.

In certain embodiments of the cannulas described herein, the guide limits the insertion angle between the axis of the cannula and the surface of the patient's skin ranging from 5° to 90°.

### BRIEF DESCRIPTION OF THE DRAWINGS

FIG. 1 shows an embodiment of a cannula described herein, where the cannula is attached to the syringe by means of a removeable attachment.

FIG. 2 shows an embodiment of a cannula that is permanently attached to the syringe.

FIG. 3 shows a detail an embodiment of a cannula with a single orifice.

FIG. 4 shows an embodiment of a cannula with a rectangular orifice.

FIG. 5 shows an embodiment of a cannula with a polygonal orifice wherein the long axis of the polygon is generally oriented at an angle α to the axis of the cannula.

FIG. 6 shows an embodiment of a cannula with a polygonal orifice wherein the polygon is generally oriented at an angle β to the axis of the cannula and the length of the polygon and the angle to the axis of the cannula enable the polygon to describe a helical shape around the cannula.

FIG. 7 shows an embodiment of a cannula with a polygonal orifice wherein opposing sides of the polygon are not parallel to each other.

FIG. 8 shows an embodiment of a portion of the perimeter of the orifice intersects with the tip of the cannula.

FIG. 9 shows an embodiment of a cannula with transverse orifices wherein the orifices are located coaxially.

FIG. 10 shows an embodiment of a cannula wherein the axes of transverse orifices are be non-perpendicular to the axis of the cannula.

FIG. 11 shows an embodiment of a cannula wherein the orifices are equally-spaced along the length of the cannula.

FIG. 12 shows an embodiment of a cannula wherein spacing between orifices increases along the length of the cannula from the distal end to the proximal end.

FIG. 13 shows an embodiment of a cannula wherein spacing between orifices increases along the length of the cannula from the proximal end to the distal end.

FIG. 14 shows an embodiment of a cannula wherein the orifices are oriented such that their axes are in a single plane that intersects the axis of the cannula.

FIG. 15 shows an embodiment of a cannula wherein the orifices are oriented such that their axes are each located in planes that are oriented in a range of angles relative to one another.

FIG. 16 shows an embodiment of a cannula wherein the cross-sectional area of each orifice varies based on its location relative to the distal end of the cannula.

FIG. 17 shows an embodiment of a cannula wherein the cross-sectional area of each orifice varies based on its location relative to the distal end of the cannula.

FIG. 18A shows a view of a cannula wherein the cannula has a ground bevel tip.

FIG. 18B shows a magnified section view of the embodiment of a cannula shown in FIG. 18A with a plugged distal end and a ground bevel tip.

FIG. 19A shows a view of an embodiment a cannula wherein the distal end is ground to an axially symmetrical point.

FIG. 19B shows a magnified section view of the embodiment of a cannula shown in FIG. 19A with a plugged distal end and a tip ground to an axially symmetrical point.

FIG. 20 shows a magnified section view of the embodiment of a cannula with an occluded distal end and a tip ground to a point that is offset from the axis of the cannula.

FIG. 21 shows an embodiment of an insertion guide that attaches to the skin and guides the insertion of the cannula to the desired depth at the desired angle.

FIG. 22 shows a cross-section of the insertion guide, syringe, and cannula, wherein the location and angle of the syringe by the guide are illustrated,

FIG. 23 shows an embodiment of an insertion guide for the cannula alone, wherein the guide controls the angle and depth of insertion of the cannula.

FIG. 24 shows a cross-section of the insertion guide of FIG. 24, showing the interaction between the guide and cannula, and showing schematically the connection between the cannula and syringe pump.

FIG. 25 shows an embodiment of the cannula, wherein the inner diameter of the cannula is gradually tapered along the entire length of the cannula.

FIG. 26 shows an embodiment wherein the internal diameter of the cannula increases along the entire length of cannula.

FIG. 27 shows an embodiment wherein the internal diameter of cannula increases in more than one discrete portions of cannula.

FIG. 28 is a graph which shows injection pressure, measured in Volts, over time of Omnipaque material injected into minipigs using three cannulas described herein compared to a standard 25 gauge needle.

### DETAILED DESCRIPTION

### Definitions

For the purposes of clarity and consistency, the term “proximal” refers to a direction away from the body of the patient and towards the cannula. The term “distal” refers to a direction towards the body of the patient and away from the cannula.

As used herein, unless otherwise noted, “axis of the cannula” refers to the long axis of the cannula, i.e. the axis running from the proximal end to the distal end.

As used herein, “orifice” means an opening in the distal end of a cannula or through the wall of a cannula for fluid discharge from the cannula.

As used herein, “oriented parallel” means that the axis of an orifice is parallel to the axis of the cannula.

As used herein, “polygon” means a plane figure with at least three straight sides and angles.

As used herein, “elongated polygon” means that one or more sides of a polygon is significantly longer than the remaining sides of the polygon.

Described herein, is a cannula for injecting medicament into a patient through at least one orifice located on the cannula wall to allow for broader and/or more even distribution of a medicament compared to a traditional cannula having an opening at the distal tip of the cannula. Orifices can be created in the cannula wall of a cannula in multiple forms for improving distribution of a medicament in tissue. Specific requirements based on the location of the injection, the depth of insertion, and the properties of the fluid to be injected can influence the number and configuration of the orifices.

Specifically, described herein, is a cannula for the dispensing of medicaments, wherein the cannula has a cannula wall, a distal end and a proximal end and wherein the cannula comprises a single orifice in the cannula wall, proximal to the distal end of the cannula or two or more orifices in the cannula wall, proximal to the distal end of the cannula. Also, described herein, is a cannula for the dispensing of medicaments, wherein the cannula has a cannula wall, a distal end and a proximal end and wherein the cannula comprises a single orifice in the cannula wall, proximal to the distal end of the cannula or two or more orifices in the cannula wall, proximal to the distal end of the cannula, wherein the longest axes of the two or more orifices are not oriented parallel to the long axis of the cannula. Also, described herein, is a cannula for the dispensing of medicaments, wherein the cannula has a cannula wall, a distal end and a proximal end and wherein the cannula comprises a single orifice in the cannula wall, proximal to the distal end of the cannula or two or more orifices in the cannula wall, proximal to the distal end of the cannula, wherein the shortest axes of the two or more orifices are not oriented parallel to the long axis of the cannula. In certain embodiments described herein, the cannula, for the dispensing of medicaments, has a cannula wall, a distal end and a proximal end and wherein the cannula comprises a single orifice in the cannula wall, proximal to the distal end of the cannula.

Whether there is a single orifice or two or more orifices in the cannula wall of the cannulas described herein, the orifices can be any shape that allows the desired dispensing of the medicament. Suitable orifice shapes can include, but are not limited to, polygonal shapes such as square, rectangle, rhombus, quadrilateral, pentagonal; circular, such as circle, ellipse, or oval; helical; or any non-symmetrical shape to allow the desired dispensing of the medicament. In certain embodiments, the single orifice or at least one of the two or more orifices in the cannula wall of the cannulas described herein can be a circular hole. In certain embodiments, the single orifice or at least one of the two or more orifices in the cannula wall of the cannulas described herein can be an ellipse. In certain embodiments, the single orifice or at least one of the two or more orifices in the cannula wall of the cannulas described herein can be a polygon. In certain embodiments, the single orifice or at least one of the two or more orifices in the cannula wall of the cannulas described herein can be an elongated polygon. In certain embodiments, the single orifice or at least one of the two or more orifices in the cannula wall of the cannulas described herein can be an elongated polygon; wherein the elongated polygon spans between 1% to 80% of the length of the cannula.

In the embodiments wherein the cannula has two or more orifices in the cannula wall, the two or more orifices can be different geometric shapes, including but not limited to, a combination of circular, elliptical, and polygonal shapes.

In the embodiments wherein the cannula has two or more orifices in the cannula wall, the two or more orifices can be spaced an equal distance apart along the axis of the cannula. In another embodiment, wherein the cannula has two or more orifices in the cannula wall, the two or more orifices can be unevenly spaced along the axis of the cannula. In other embodiments, the two or more orifices are spaced at increasing distances apart in the distal direction along the axis of the cannula. In still other embodiments, the two or more orifices are spaced at decreasing distances apart in the distal direction along the axis of the cannula.

In the embodiments wherein the cannula has two or more orifices in the cannula wall, the two or more orifices are not aligned in a column along the length of the cannula. In certain embodiments, wherein the cannula has two or more orifices in the cannula wall, the two or more orifices are distributed around the circumference of the cannula and varying distances from each other. In certain embodiments, wherein the cannula has two or more orifices in the cannula wall, the two or more orifices are unevenly distributed along the circumference of the cannula. In certain embodiments, wherein the cannula has two or more orifices in the cannula wall, the two or more orifices are unevenly distributed along the circumference of the cannula having varying distances from each other.

Whether there is a single orifice or two or more orifices in the cannula wall of the cannulas described herein, the orifices can be any size that allows the desired dispensing of the medicament. Orifice area is a function of several factors, including but not limited to the volume of medicament dispensed, the viscosity of the medicament, the inner diameter of the cannula, and the process used to generate the orifices. A practical limit for the diameter, minor axis, or shortest side of an orifice is 0.002 inches. A practical upper limit for diameter of an orifice, the minor or major axis of an orifice, or the side of a polygon-shaped orifice generally perpendicular to the axis of the cannula is 75% of the outer diameter of the cannula. Whether there is a single orifice or two or more orifices in the cannula wall of the cannulas described herein, the orifices can each comprise an area between 0.000001 sq. in. to 0.015 sq. in.

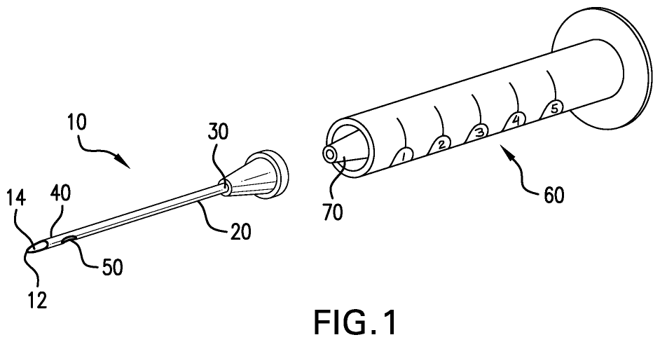

In certain embodiments, as shown in FIG. 1, the cannula 10 has a cannula wall 20 a proximal end 30 and a distal end 40. In the cannula shown in FIG. 1, the cannula 10 has a single orifice 50 in the cannula wall 30, proximal to the distal end 40. Also as shown in FIG. 1, the distal end 40 of cannula 10 comprises a typical sharpened ground bevel tip 12. In certain embodiments, the distal end 40 of cannula 10 would comprise a distal opening 14 for additional discharge of medicament, in addition to the orifice 50.

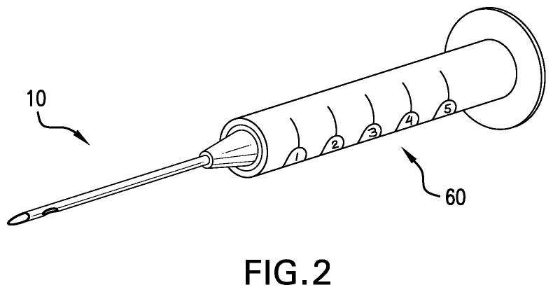

As shown in FIG. 1, the cannula 10 can be attached to a syringe 60. There are several typical methods of attaching a cannula to a syringe. In one embodiment, the cannula can be removably attached to a syringe. In the embodiment shown in FIG. 1, cannula 10 is removably attached to a syringe 60 by Luer connection 70. In alternate embodiments, other attachment means would comprise, but not be limited to, a threaded connection. In other embodiments, the cannula can be permanently attached to or an integral part of the syringe. In alternate embodiments, cannula 10 would be permanently attached to syringe 60, as shown in FIG. 2.

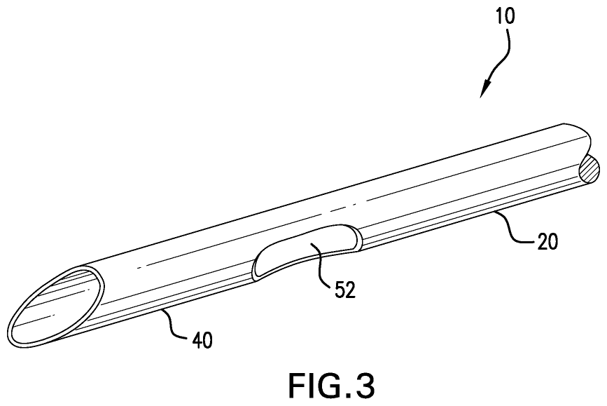

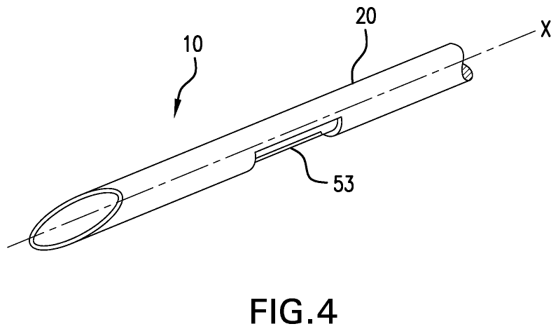

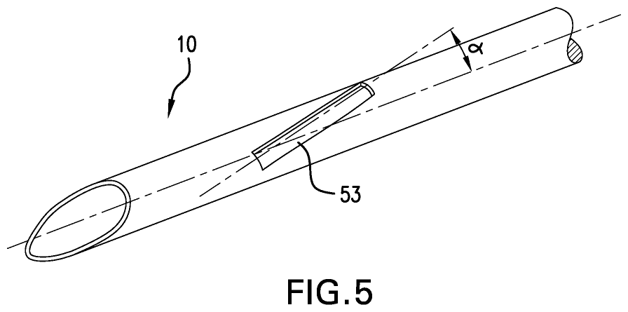

In the embodiment shown in FIG. 1, the orifice 50 is circular in shape. In alternate embodiments, the orifice can be a different shape. FIG. 3, shows a cannula 10 with a cannula wall 20, wherein the cannula wall 20 has an orifice 52 that is elliptical in shape, proximal to the distal end 40 of the cannula. In other embodiments, the orifice can be polygonal in shape. FIG. 4, shows a cannula 10 with a cannula wall 20, wherein the cannula wall 20 has an orifice 53 that is polygonal, having a rectangular shape. Such a rectangular orifice 53 may be parallel to the axis of the cannula, as shown in FIG. 4, or oriented on a slight angle α to the axis of the cannula 10, as shown in FIG. 5.

In certain embodiments of the cannula described herein, wherein the cannula wall contains one or more than two orifices, the orifice or orifices can be oriented at an angle to the axis of the cannula between, and including, 5° to 90°. In certain embodiments, the orifice or orifices can be oriented at a 5° angle to the axis of the cannula. In certain embodiments, the orifice or orifices can be oriented at a 10° angle to the axis of the cannula. In certain embodiments, the orifice or orifices can be oriented at a 15° angle to the axis of the cannula. In certain embodiments, the orifice or orifices can be oriented at a 20° angle to the axis of the cannula. In certain embodiments, the orifice or orifices can be oriented at a 25° angle to the axis of the cannula. In certain embodiments, the orifice or orifices can be oriented at a 30° angle to the axis of the cannula. In certain embodiments, the orifice or orifices can be oriented at a 35° angle to the axis of the cannula. In certain embodiments, the orifice or orifices can be oriented at a 40° angle to the axis of the cannula. In certain embodiments, the orifice or orifices can be oriented at a 45° angle to the axis of the cannula. In certain embodiments, the orifice or orifices can be oriented at a 50° angle to the axis of the cannula. In certain embodiments, the orifice or orifices can be oriented at a 55° angle to the axis of the cannula. In certain embodiments, the orifice or orifices can be oriented at a 60° angle to the axis of the cannula. In certain embodiments, the orifice or orifices can be oriented at a 65° angle to the axis of the cannula. In certain embodiments, the orifice or orifices can be oriented at a 70° angle to the axis of the cannula. In certain embodiments, the orifice or orifices can be oriented at a 75° angle to the axis of the cannula. In certain embodiments, the orifice or orifices can be oriented at an 80° angle to the axis of the cannula. In certain embodiments, the orifice or orifices can be oriented at a 85° angle to the axis of the cannula. In certain embodiments, the orifice or orifices can be oriented at a 90° angle to the axis of the cannula.

In certain embodiments, the cannula described herein comprises a cannula wall comprising a single orifice or two or more orifices, wherein the single orifice or at least one of the two or more of the orifices is shaped as an elongated polygon, wherein the longest axis of the elongated polygon is oriented at an angle to the axis of the cannula between 0° and 45°.

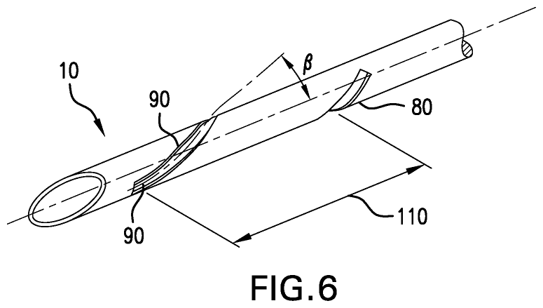

In an alternate embodiment, orifices may be oriented such that their axes are not in a single plane 110 that intersects the axis of cannula 10, as shown in FIG. 6. A rectangular orifice 54 may have an angle β between its long edges 80 and the axis of the cannula 10 wherein the orifice is sufficiently long that it acquires a helical shape 90, as shown in FIG. 6, wherein the pitch of the helix is a function of the angle β and the diameter of the cannula 10.

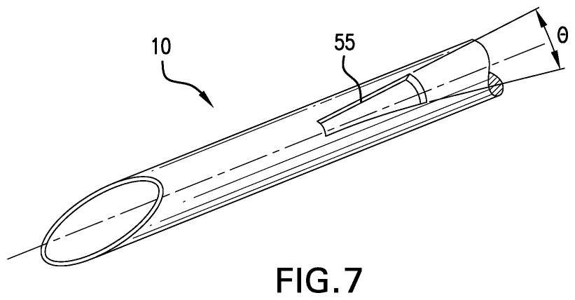

In alternate embodiments, polygonal orifices with long edges may comprise an angle θ between the two long edges of the polygon, wherein the polygon results in an orifice 55 that widens along its length, as shown in FIG. 7.

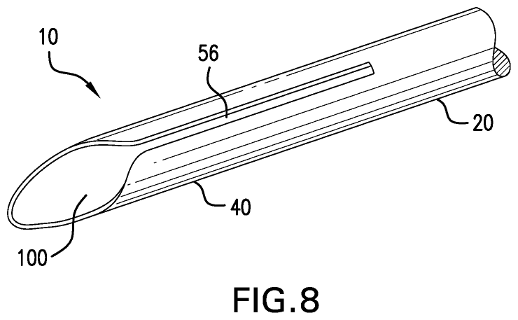

In alternate embodiments, the orifice is not completely defined by the cannula wall. In certain embodiments, a portion of the perimeter of an orifice intersects with the distal end of the cannula. In certain embodiment, a cannula 10 having a cannula wall 20 comprises an orifice 56 wherein, a portion of the perimeter of the orifice intersects with the cannula tip 100 located at the distal end 40 of the cannula 10, as shown in FIG. 8.

Also described herein, is a cannula for the dispensing of medicaments, wherein the cannula has a cannula wall, a distal end and a proximal end and wherein the cannula comprises two or more orifices in the cannula wall, proximal to the distal end of the cannula, wherein the two or more orifices are not oriented parallel to the axis of the cannula.

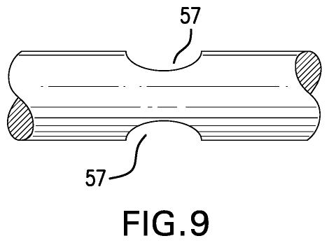

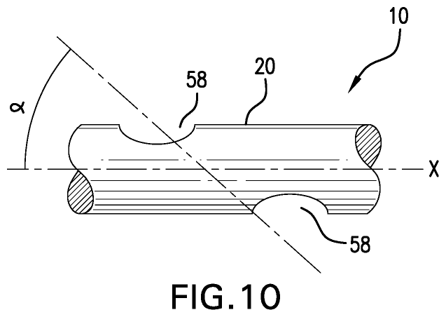

In certain embodiments, as shown in FIG. 9, transverse orifices 57 are located directly opposite from each other, such that two diametrically opposed openings would be created. In an alternate embodiment, shown in FIG. 10, transverse orifices 58 of cannula 10 are not located directly opposite each other but are offset from each other.

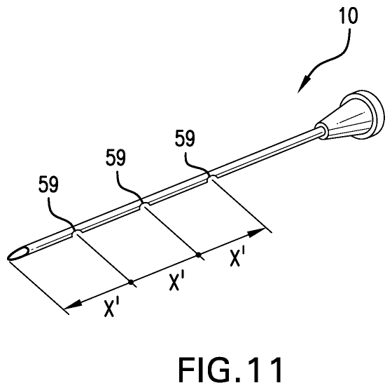

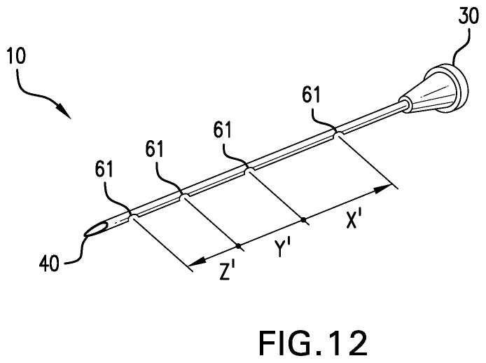

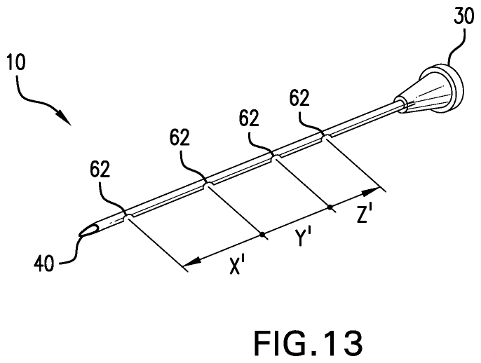

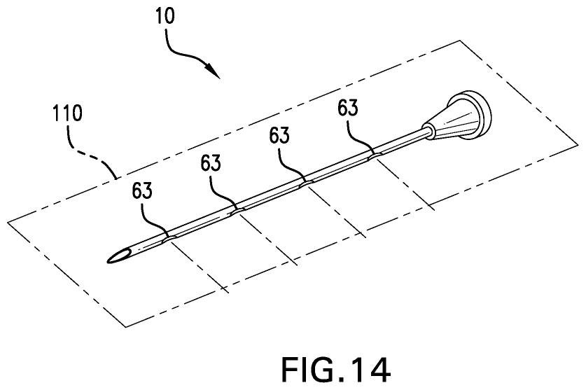

In another embodiment, orifices 59 may be created wherein they are equally-spaced along the length of the cannula 10, as shown in FIG. 11 (distance is shown as X′). In an alternate embodiment, spacing of orifices 61 may vary according to a schedule wherein spacing between orifices 61 increases along the length of the cannula 10 from distal end 40 to the proximal end 30, as shown in FIG. 12 (different lengths shown as Z′, Y′ and X′). Alternately, spacing of openings may vary according to a schedule wherein spacing between orifices 62 decreases along the length of the cannula 10 from distal end 40 to proximal end 30, as shown in FIG. 13. As depicted in FIGS. 13 and 14, X′ represents the longest length of space between two orifices, Z′ represents the shortest length of space between to orifices and Y′ represents a length of space between Z′ and X′.

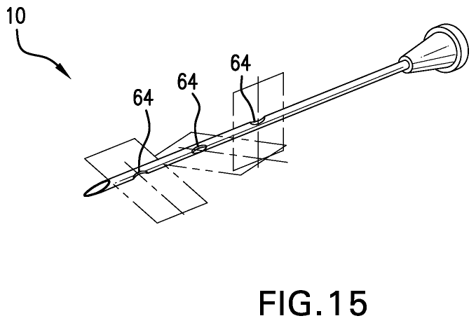

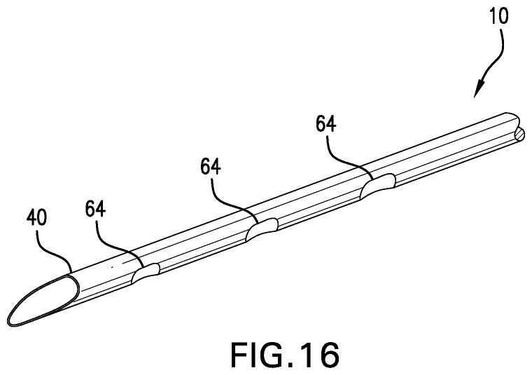

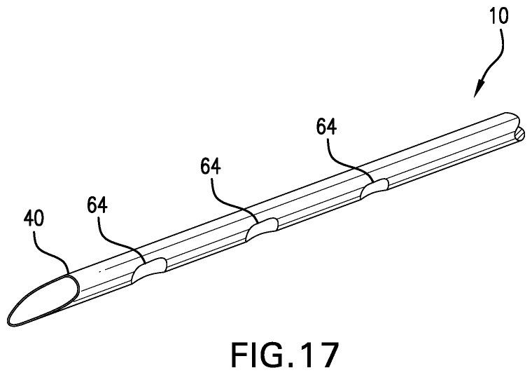

In yet another embodiment, all orifices 63 may be oriented such that their axes are in a single plane 110 that intersects the axis of cannula 10, as shown in FIG. 14. In an alternate embodiment, the axes of orifices 64 may be located at a range of angles relative to one another, as shown in FIG. 15. In yet another embodiment, the cross-sectional area of the orifices may be constant for all orifices. In an alternate embodiment, the cross-sectional area of each orifices 64 may vary based on its location on the cannula 10, as shown in FIG. 16 and FIG. 17. In FIG. 16, the diameter of the orifices increases from the distal to the proximal end. In FIG. 17, the diameter of the orifices decreases from the distal to the proximal end.

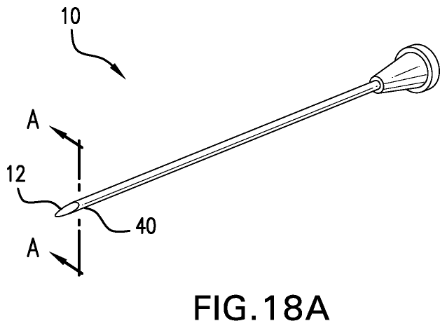

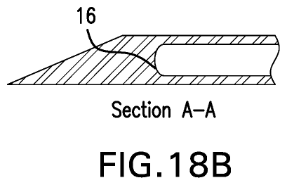

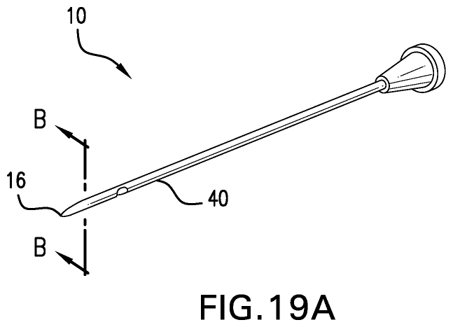

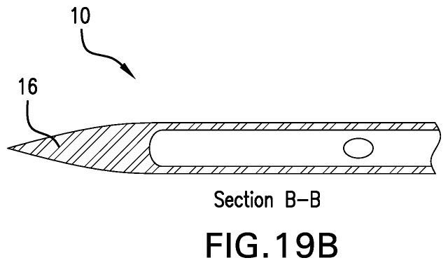

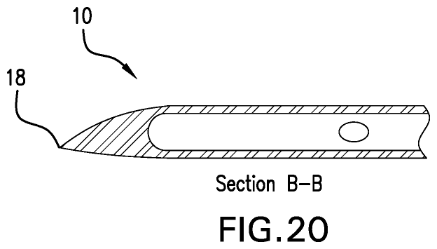

In certain embodiments described herein, the cannula can be augmented such that the orifices in the cannula wall are the only means of distributing medicament. For example, as in FIG. 18A and FIG. 18B, the distal end 40 of cannula 10 comprises a sharpened ground bevel tip 12 comprising an occluded distal end 16 to prevent flow of medicament thru the distal end 40 of the cannula 10. During some procedures the occluded tip may be used to locate the intramuscular layer, ensuring that the cannula and the orifices do not enter the intramuscular layer. Thus, when the medicament is dispersed through the orifices, the medicament is dispersed only within the subcutaneous layer. Such an occlusion can be achieved by, but not be limited to, welding means, including laser welding means, crimping means, or plug means. Typically, such occlusions would be created prior to sharpening the distal end 40 of cannula 10. In an alternate embodiment, distal end 40 of cannula 10 can be occluded and ground to an axially symmetrical point 16, as shown in FIG. 19A and FIG. 19B. In alternate embodiments the axis of the tip 18 would not coincide with the axis of the cannula 10, as shown in FIG. 20.

In many applications, it is critical for the medicament to be distributed in a specific layer of tissue, such as subcutaneous or intramuscular. In such applications the depth of fluid dispersion is instrumental to optimized delivery of medicament. Control of the angle and depth of cannula insertion is critical for correct positioning of the orifice or orifices in the cannula wall. Use of a guide in combination with the cannula described herein can aid in controlling the angle and depth of cannula insertion. In certain embodiments, the cannula described herein further comprises a guide for controlling the angle and depth of insertion of the cannula. In certain embodiments, the guide can limit the insertion depths of the distal end of the cannula between ¼″ and 3″. In certain embodiments, the guide can limit the insertion angle between the axis of the cannula and the surface of the patient's skin ranging from 5° to 90°.

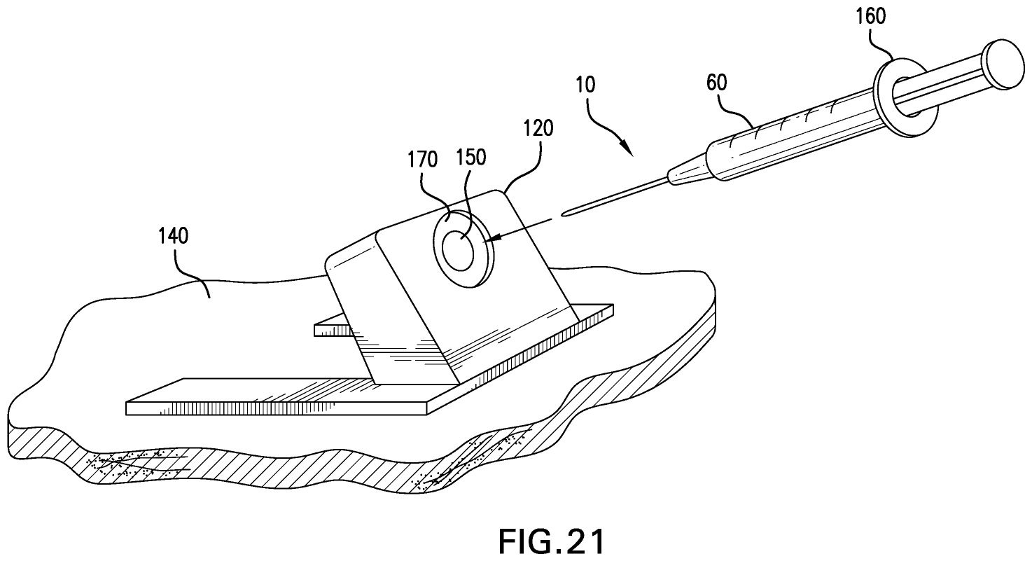

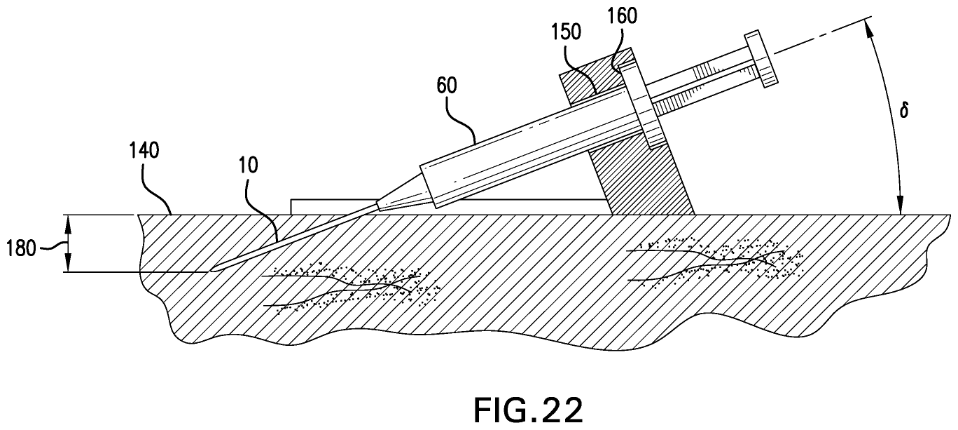

In one embodiment, as shown in FIG. 21, an insertion fixture, or guide 120 attaches to skin 140 at the location of insertion. Guide 120 comprises a linear opening 150 to accept the cannula 10 and syringe 60. In one embodiment, cannula 10 is inserted to the full depth of opening 150 until the flange 160 of syringe 60 contacts a stop 170 adjacent to linear opening 150. The location of opening 150, the location of the stop 170, the length of cannula 10, and the angle δ between the axis of opening 150 and skin 140 control the depth of insertion 180, this is shown in FIG. 22.

The guide may be constructed of a variety of materials, most notably metals and plastics. In one embodiment, the guide may be machined, cast, or 3D printed in an appropriate metal including, but not limited to, stainless steel or titanium. In an alternate embodiment, the guide would be thermoformed from a variety of plastics including, but not limited to, PETG, Polypropylene, or ABS. In a preferred embodiment, the guide would be injection molded from a variety of plastic resins, including, but not limited to, ABS, Polypropylene, or Polyethylene.

In certain embodiments, the guide may be affixed to the skin of the patient by means of one or more straps that encompass the circumference of an extremity, such as an arm or leg. In an alternate embodiment, the guide could be affixed to the patient's skin by means of a pressure sensitive adhesive. Such adhesive could be selected from a group that includes, but is not be limited to, acrylic, hydrocolloid, urethane, or silicone adhesives.

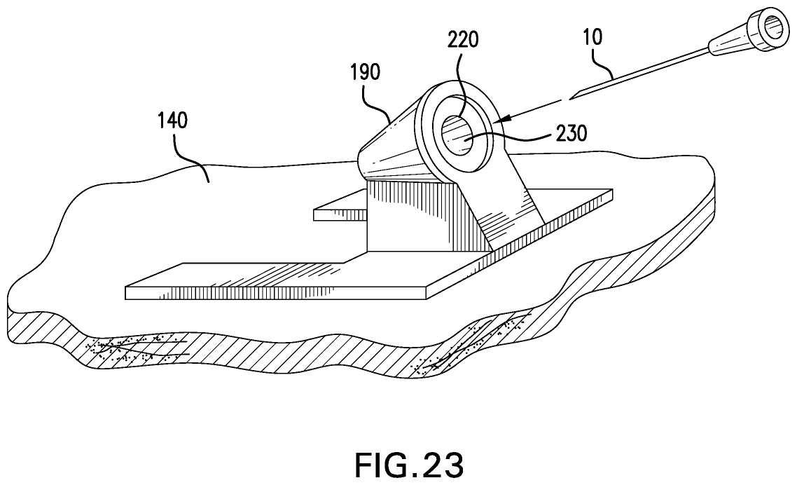

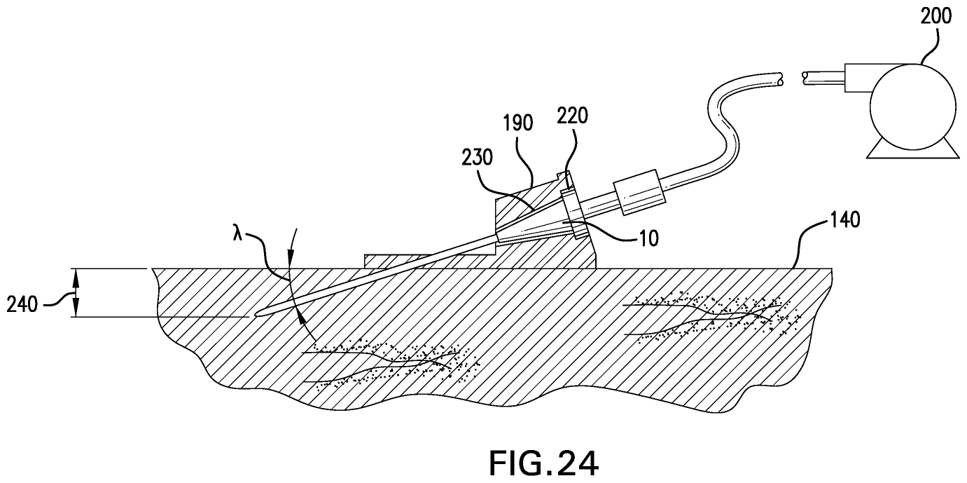

It may be desirable, in cases where the delivery of medicament encompasses a long period of time or the viscosity of the medicament is sufficiently high to make delivery by means of a manually operated syringe difficult, a separate pump 200, such as an infusion pump or a syringe pump may be used to displace medicament into the cannula 10. In such an embodiment, a smaller guide 190 may be used to locate cannula 10 in place without a syringe. The location of the stop 220 of the smaller guide 190 and the angle λ, between the axis of opening 230 and skin 140, and the length of cannula 10 would determine the depth of insertion 240 of cannula 10, as shown in FIG. 23 and FIG. 24.

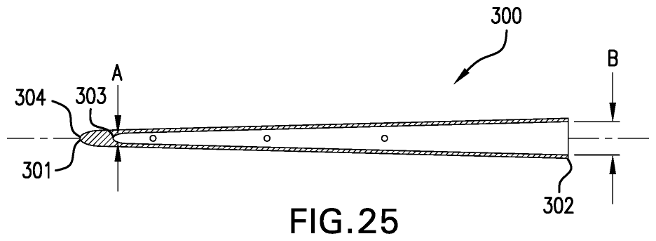

In certain embodiments of the cannulas described herein, the inner diameter of the cannula is not constant along the length of the cannula. In certain embodiments, the inner cannulas described herein, are tapered. Regarding the cannulas described herein, “tapered” means that the inner diameter of the distal end of the cannula is smaller than the inner diameter of the proximal end of the cannula. To reduce resistance to flow of fluid through the cannula, the inner diameter may increase in diameter from the inner diameter at the distal end to the inner diameter at the proximal end. As shown in FIG. 25, the inner diameter A of cannula 300 at the distal end 301 increases in size to diameter B at the proximal end 302 of the cannula 300.

The increase in diameter from the distal end of the cannula to the proximal may have a range from an inner diameter of “X” mm at the distal end to an inner diameter of between 1.25X mm to 5X at the proximal end. In certain embodiments, the increase in diameter from the distal end of the cannula to the proximal may have a range from an inner diameter of “X” mm at the distal end to an inner diameter of 5X mm at the proximal end. In certain embodiments, the increase in diameter from the distal end of the cannula to the proximal may have a range from an inner diameter of “X” mm at the distal end to an inner diameter of 4.75X mm at the proximal end. In certain embodiments, the increase in diameter from the distal end of the cannula to the proximal may have a range from an inner diameter of “X” mm at the distal end to an inner diameter of 4.5X mm at the proximal end. In certain embodiments, the increase in diameter from the distal end of the cannula to the proximal may have a range from an inner diameter of “X” mm at the distal end to an inner diameter of 4.25X mm at the proximal end. In certain embodiments, the increase in diameter from the distal end of the cannula to the proximal may have a range from an inner diameter of “X” mm at the distal end to an inner diameter of 4X mm at the proximal end. In certain embodiments, the increase in diameter from the distal end of the cannula to the proximal may have a range from an inner diameter of “X” mm at the distal end to an inner diameter of 3.75X mm at the proximal end. In certain embodiments, the increase in diameter from the distal end of the cannula to the proximal may have a range from an inner diameter of “X” mm at the distal end to an inner diameter of 3.5X mm at the proximal end. In certain embodiments, the increase in diameter from the distal end of the cannula to the proximal may have a range from an inner diameter of “X” mm at the distal end to an inner diameter of 3.25X mm at the proximal end. In certain embodiments, the increase in diameter from the distal end of the cannula to the proximal may have a range from an inner diameter of “X” mm at the distal end to an inner diameter of 3X mm at the proximal end. In certain embodiments, the increase in diameter from the distal end of the cannula to the proximal may have a range from an inner diameter of “X” mm at the distal end to an inner diameter of 2.75X mm at the proximal end. In certain embodiments, the increase in diameter from the distal end of the cannula to the proximal may have a range from an inner diameter of “X” mm at the distal end to an inner diameter of 2.5X mm at the proximal end. In certain embodiments, the increase in diameter from the distal end of the cannula to the proximal may have a range from an inner diameter of “X” mm at the distal end to an inner diameter of 2.25X mm at the proximal end. In certain embodiments, the increase in diameter from the distal end of the cannula to the proximal may have a range from an inner diameter of “X” mm at the distal end to an inner diameter of 2X mm at the proximal end. increase in diameter from the distal end of the cannula to the proximal may have a range from an inner diameter of “X” mm at the distal end to an inner diameter of 1.75X mm at the proximal end. In certain embodiments, the increase in diameter from the distal end of the cannula to the proximal may have a range from an inner diameter of “X” mm at the distal end to an inner diameter of 1.5X mm at the proximal end. In certain embodiments, the increase in diameter from the distal end of the cannula to the proximal may have a range from an inner diameter of “X” mm at the distal end to an inner diameter of 1.25X mm at the proximal end.

In certain embodiments, value of “X” as the inner diameter of the cannula at the distal end can be the minimum inner diameter of a 10-34 gauge needle according to the Stainless Steel Needle Tubing for the Manufacture of Medical Devices—Requirements and test methods (ISO 9626:2016), incorporated herein by reference.

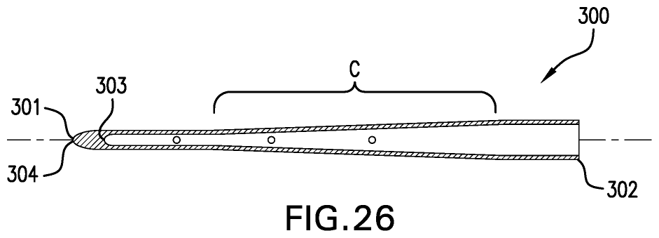

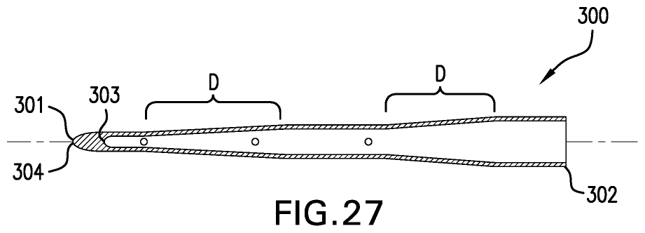

As shown in FIG. 25 cannula 300 is gradually tapered along the entire length of the cannula. FIG. 26 shows an embodiment wherein the internal diameter of cannula 300 increases along a portion C of the length of cannula 300. FIG. 27 shows an embodiment wherein the internal diameter of cannula 300 increases in more than one discrete portions D along the length of cannula 300.

Additionally, FIGS. 25 through 27 show a cannula 300 with a plugged distal end 303 and a tip 304 ground to an axially symmetrical point. Alternately, the cannula can have an unplugged distal end and any type of point described herein may be employed with a tapered cannula embodiment.

Also disclosed herein are processes of making and manufacturing the cannulas described herein. Described herein are processes of manufacturing the cannulas described herein wherein orifices are laser drilled or machine drilled into a suitably configured cannula. In certain embodiments, a suitable-sized cannula is laser drilled. A laser may be used to create orifices of desired sizes and shapes, with orifice widths ranging as small as 0.002 inches. It can be appreciated that such a precise means for cutting through the cannula wall can be utilized to create orifices of any shape determined to be beneficial to cannula performance. The depth of such cuts can be controlled by altering laser power and cutting speed to create an orifice on one cannula wall, or through both opposing walls.

Alternatively, Electrical discharge machining (EDM) may be employed to produce precise small orifices of various shapes through one wall or both walls of a cannula. Mechanical drilling may also be employed to pierce one wall of the cannula or both opposing walls. However with mechanical drilling orifices are limited to circular shapes and orifice sizes may be larger than those that can be achieved with laser cutting or Wire Electro Discharge Machining. Milling and grinding may also be employed to produce non-round orifice shapes, but with minimum orifice sizes again limited by the physical size of cutting tools. A variety of cutting means can therefore be employed to create orifices in a cannula, with the method of choice guided by speed, cost, and orifice shape requirements.

### EXAMPLES

### Example 1

Minipigs were injected with 4.5 ml of Omnipaque material at a rate of 10 ml/min using each of the described cannulas below:

| | Length | | |
|---|---|---|---|
| Needle | (mm) | Qty | Configuration |
| 10 | 31.75 | 49 | Planar thru hole arrangement; Hole spacing 9.5 mm; Hole 1 |
| | | | (proximal) Dia. 0.0043″ (0.1092); Hole 2 (middle) Dia. 0.0043″ |
| | | | (0.1092 mm); Hole 3 (distal) Dia. 0.0043″ (0.1092) |
| 21 | 12.7 | 55 | Three sets of thru holes, 4 mm, 7 mm, and 10 mm from hub; Holes |
| | | | oriented 120° apart from each other. Hole 1 Dia. 0.0043″ (0.1092); |
| | | | Hole 2 Dia. 0.0043″ (0.1092); Hole 3 Dia. 0.0043″ (0.1092) |
| 30 | 31.75 | 52 | Tapered Needle; outer radius = 0.116 mm; inner radius = 0.23 mm; |
| | | | Hole spacing 9.5 mm ; Hole 1 (proximal) Dia. 0.0043″ (0.1092); Hole |
| | | | 2 (middle) Dia. 0.0043″ (0.1092 mm); Hole 3 (distal) Dia. 0.0043″ |
| | | | (0.1092) |
| 40 | 31.75 | 51 | Standard 25 gauge needle. Not cross drilled. Open End. Chisel Point. |

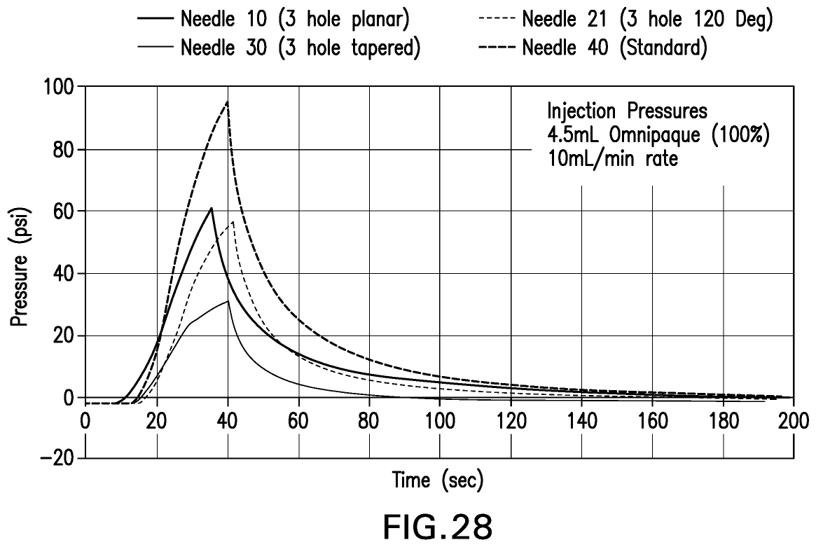

FIG. 28 shows the injection pressure, measured in PSI, over time of Omnipaque material injected into minipigs using Needles 10, 21 and 30 described herein compared to a standard 25 gauge needle. As shown in FIG. 28, Needles 10, 21 and 30 all exhibited reduced injection pressure as compared to Needle 40, a standard 25 gauge needle.

1. A cannula for subcutaneous dispensing of medicaments, wherein the cannula has a cannula wall, a distal end and a proximal end and wherein the cannula comprises a single orifice in the cannula wall, proximal to the distal end of the cannula or two or more orifices in the cannula wall, proximal to the distal end of the cannula, wherein the long axes of each of the two or more orifices are not parallel to the axis of the cannula.

2. (canceled)

3. The cannula of claim 1, wherein the two or more orifices are oriented at an angle to the axis of the cannula between 5° and 90°.

4. (canceled)

5. (canceled)

6. (canceled)

7. (canceled)

8. (canceled)

9. The cannula of claim 1, wherein the single orifice or the two or more orifices are in the shape of an ellipse.

10. The cannula of claim 1, wherein the single orifice or the two or more orifices are in the shape of a polygon.

11. The cannula of claim 10, wherein the polygon is an elongated polygon.

12. The cannula of claim 11, wherein the length of the elongated polygon spans range between 1% and 80% of the length of the cannula.

13. The cannula of claim 12, wherein the long axis of the elongated polygon is oriented at an angle to the axis of the cannula between 0° and 45°.

14. The cannula of claim 1, wherein the single orifice or the two or more orifices are not closed such that a portion of the perimeter of the opening of the orifice or two or more orifices intersects with the distal end of the cannula.

15. The cannula of claim 1, wherein the two or more orifices are oriented at a range of angles from one another, wherein the angles range from 0° to 180° when viewed in cross section along the axis of the cannula.

16. The cannula of claim 1, wherein the two or more orifices are spaced an unequal distance apart along the axis of the cannula.

17. The cannula of claim 1, wherein the two or more orifices are spaced at increasing distances apart in the distal direction along the axis of the cannula.

18. The cannula of claim 1, wherein the two or more orifices are spaced at decreasing distances apart in the distal direction along the axis of the cannula.

19. The cannula of claim 1, wherein the two or more orifices comprise a combination of elliptical and polygonal shapes.

20. The cannula of claim 1, wherein the single orifice or the two or more orifices each comprise an area between 0.000001 sq. in to 0 0.015 sq. in.

21. The cannula of claim 1, wherein the inner diameter of the cannula increases from the distal end of the cannula to the proximal end.

22. The cannula of claim 21, wherein the increase in inner diameter occurs along the entire length of the cannula.

23. The cannula of claim 21, wherein the increase in inner diameter occurs along one or more discrete portions of the length of the cannula.

24. The cannula for the dispensing of medicaments of claim 1, wherein the cannula further comprises a guide for controlling the angle and depth of insertion of the cannula.

25. The guide of claim 24, wherein the cannula is limited to a range of insertion depths of the distal end of the cannula between ¼ inch and 3 inches.

26. The guide of claim 24, wherein the cannula is limited to an insertion angle between the axis of the cannula and the surface of the patient's skin ranging from 5° to 90°.
